(19) United States
(12) Patent Application Publication
(10) Pub. No.: US 2022/0323254 A1
(43) Pub. Date: Oct. 13, 2022

(54) ABLATION DEVICE FOR THE REDUCTION OF INTRAOCULAR PRESSURE AND METHODS OF USE

(71) Applicant: Innovative Drive Corporation, Reno, NV (US)

(72) Inventors: Luke W. Clauson, Reno, NV (US); Matthew B. Newell, Reno, NV (US); Brendan Reese, Reno, NV (US)

(21) Appl. No.: 17/716,884

(22) Filed: Apr. 8, 2022

Related U.S. Application Data
(60) Provisional application No. 63/173,225, filed on Apr. 9, 2021.

(51) Int. Cl. A61F 9/007

(52) U.S. Cl. CPC A61F 9/00781; A61B 2017/320069

(57) ABSTRACT

An ablation device for the treatment of an eye to lower intraocular pressure including a handle; and an array of rods projecting from a distal end region of the handle and electrically-connected to an energy delivery generator. Each rod of the array of rods is configured to create a cavity in a surface of the eye via ab externo tissue ablation to enhance drainage of aqueous humor from the eye. Related devices, systems, and methods are provided.

### CROSS-REFERENCE TO RELATED APPLICATIONS

This application claims the benefit of priority under 35 U.S.C. § 119(e) to co-pending U.S. Provisional Patent Application Ser. No. 63/173,225, filed Apr. 9, 2021. The disclosure of the application is incorporated by reference in its entirety.

### BACKGROUND

Glaucoma is a complicated disease in which damage to the optic nerve leads to progressive vision loss and is the leading cause of irreversible blindness. Aqueous humor is the fluid that fills the anterior chamber in front of the iris and the posterior chamber of the eye behind the iris. Vitreous humor or vitreous body is a gel-like material found in the posterior segment of the eye posterior of the capsular bag. FIG. 1A is a diagram of the front portion of an eye 5 showing the lens 7, cornea 8, iris 9, ciliary body 6 including ciliary processes 4, trabecular meshwork 10, and Schlemm's canal 12. The aqueous humor is a fluid produced by the ciliary body 6 that lies behind the iris 9 adjacent to the lens 7. This aqueous humor washes over the lens 7 and iris 9 and flows to the drainage system located in the angle of the anterior chamber. The angle of the anterior chamber, which extends circumferentially around the eye, contains structures that allow the aqueous humor to drain. Aqueous humor is absorbed through the trabecular meshwork 10 into Schlemm's canal 12 into collector channels and passing through the sclera 15 into the episcleral venous circulation. The trabecular meshwork 10 extends circumferentially around the anterior chamber 16 in the angle. The trabecular meshwork 10 limits the outflow of aqueous humor. Schlemm's canal 12 is located beyond the trabecular meshwork 10. The two arrows in the anterior chamber 16 of FIG. 1A show the flow of aqueous humor from the ciliary body 6, over the lens 7, over the iris 9, through the trabecular meshwork 10, and into Schlemm's canal 12 and its collector channels.

In some cases glaucoma is caused by blockage of aqueous humor outflow such as by sclerosis of the trabecular meshwork, pigment or membrane in the angle. In other cases, blockage is due to a closure of the angle between the iris and the cornea. This angle type of glaucoma is referred to as “angle-closure glaucoma”. In the majority of glaucoma cases, however, called “open angle glaucoma”, the cause is unknown.

Treatments of glaucoma attempt to lower intraocular pressure (TOP) pharmacologically or by surgical intervention that enhance outflow of aqueous humor through the outflow pathways. Ab externo trabeculectomy is a type of glaucoma surgery that creates a new path as a “controlled” leak for fluid inside the eye to drain out. Conventionally, a partial thickness scleral flap is formed followed by the creation of a small hole into the anterior chamber. Aqueous humor can flow into the subconjunctival space creating a filtering bleb. The scleral flap is raised up and a blade used to enter the anterior chamber. During the operation a hole is created under the scleral flap that is fluidically connected to the anterior chamber creating an opening. The opening is partially covered with the scleral flap. A small conjunctival “bleb” or bubble appears over the scleral flap, often near the junction of the cornea and the sclera (limbus).

Minimally-invasive surgical procedures provide TOP lowering by enhancing the natural drainage pathways of the eye with minimal tissue disruption. Minimally-invasive glaucoma surgery (MIGS) uses microscopic-sized equipment and tiny incisions. MIGS offers an alternative to conventional glaucoma surgeries with the potential benefit of reducing a patient's dependence on topical glaucoma medication. Trabeculectomies and trabeculotomies can each be performed ab interno, or from inside the anterior chamber. Ab interno approaches aim to decrease TOP by increasing aqueous humor outflow through a direct opening in the trabecular meshwork from within the anterior chamber so that there is direct communication between the anterior chamber and the outer wall of Schlemm's canal. Ab interno approaches include the TRABECTOME (MST/NeoMedix Corp.) electrosurgical instrument that ablates and removes trabecular meshwork, the Kahook Dual Blade (New World Medical) for excisional goniotomy removing a strip of trabecular meshwork, gonioscopy assisted transluminal trabeculotomy (GATT) involving cutting through the trabecular meshwork, cannulating Schlemm's canal, and Omni (Sight Sciences) for performing viscoplasty or trabeculotomy through an ab interno approach for cannulating Schlemm's canal. Other ab interno methods include the iStent (Glaukos) to create pathway through the trabecular meshwork for improved outflow of aqueous humor through Schlemm's canal.

Whether ab interno or ab externo surgical approach is used, some form of surgical incision is required to perform these various procedures. Incisions in the eye can lead to complications including infection, leakage, hyphema, choroidal detachment or effusion, and others.

In view of the foregoing, there is a need for improved devices and methods related to ophthalmic surgery for the treatment of glaucoma.

### SUMMARY

In an aspect, described is an ablation device for the treatment of an eye to lower intraocular pressure including a handle; and an array of rods projecting from a distal end region of the handle and electrically-connected to an energy delivery generator. Each rod of the array of rods is configured to create a cavity in a surface of the eye via ab externo tissue ablation to enhance drainage of aqueous humor from the eye.

The surface of the eye can include the sclera. Each cavity can have a depth that is less than a full thickness of the sclera. Each cavity can be at least about 70% a thickness of the sclera up to about 90% the thickness of the sclera. At least one cavity is 100% a thickness of the sclera. Resistance to diffusion by the sclera is lowered. Each rod of the array of rods can be square-tipped or rounded in profile. A cross-section of each rod of the array of rods can be rectangular, circular, triangular, or hexagonal. The array of rods can be designed to penetrate tissue without applying energy from the energy delivery generator. Each rod of the array of rods can be fixed relative to the distal end region of the handle so that the array of rods forms a footprint having a distal concave contour that complements an external convex contour of a portion of the eye being treated. The footprint of the array can conform generally to the external convex contour when a longitudinal axis of the handle is aligned perpendicular to a point of contact with the eye.

The device can further include a gauge tool or gauge feature near a distal end of the device to align the array a selected distance from the limbus of the eye. The device can further include a depth-setting mechanism configured to control depth of insertion of the array. The depth-setting mechanism can be adjustable. The depth-setting mechanism can include an actuator that is coupled to a depth setting plate. The depth setting plate can include a plurality of openings extending through its thickness, each opening of the plurality of openings is sized and shaped to receive each rod of the array of rods so that the array of rods projects a distance distal to the plate. A position of the plate relative to the distal end region of the handle sets a length of each rod of the array of rods that is exposed distal to the plate. The actuator can be configured to incrementally change the position of the plate. Actuating the actuator to advance the depth setting plate distally can reduce an exposed length of each rod and wherein actuating the actuator to retract the depth setting plate proximally increases an exposed length of each rod. The depth setting mechanism can further include a locking mechanism configured to fix a position of the plate.

Each rod can have an exposed length available to extend within the surface of the eye. The exposed length can be greater than a thickness of the sclera of the eye. The exposed length can be adjustable. The exposed length of the rods can be adjustable such as by moving the depth setting plate relative to the rods. One or more rods of the array of rods can be hollow and usable as a flashback indicator for detecting liquid. One or more rods of the array of rods can be a depth sensor.

The energy delivery generator can be an external generator coupled to the handle by a cable. The energy delivery generator can be integrated into the handle. The energy delivery generator can be a radio frequency (RF) generator. The energy delivery generator can be integrated within a proximal, reusable portion of the handle, the reusable portion of the handle designed to operatively couple with a distal, disposable portion of the handle. The proximal, reusable portion and distal, disposable portion can couple together by threads, snap-lock, or bayonet lock. The energy delivery generator can be a monopolar RF generator or a bipolar RF generator. Each rod of the array of rods can contain two poles. The energy delivery generator can operate at a frequency in the range of about 350 kHz to 500 kHz, preferably about 490 kHz. The monopolar voltage can be 1000V or more. The bipolar voltage can be under 1000V. The energy delivery generator can use high voltage pulses. The energy delivery generator can be an ultrasonic energy generator. The energy delivery generator can use irreversible electroporation.

Each rod of the array can be stationary or movable relative to the handle so as to extend distally to penetrate tissue upon actuation. Each rod of the array of rods can be hollow for deposition of a substance into the cavity. Each rod of the array of rods can be automatically advanced to a fixed target depth. Each rod of the array of rods can be automatically advanced to a depth determined by a sensor of the device. The sensor can be a physical depth sensor or an ultrasound-enabled depth sensor. The device can further include a plurality of extendable needles, each rod of the array having an extendable needle.

In an interrelated aspect, described in an ablation system for the treatment of an eye to lower intraocular pressure including an energy delivery generator; a handle; and an array of rods projecting from a distal end region of the handle and electrically-connected to the energy delivery generator. Each rod of the array of rods is configured to create a cavity in a surface of the eye via ab externo tissue ablation to enhance drainage of aqueous humor from the eye.

The energy delivery generator can be an external generator coupled to the handle by a cable or integrated into the handle. The energy delivery generator can be a radio frequency (RF) generator. The energy delivery generator can be integrated within a proximal, reusable portion of the handle, the reusable portion of the handle designed to operatively couple with a distal, disposable portion of the handle. The distal, disposable portion can include the array of rods.

In some variations, one or more of the following can optionally be included in any feasible combination in the above methods, apparatus, devices, and systems. More details are set forth in the accompanying drawings and the description below. Other features and advantages will be apparent from the description and drawings.

### BRIEF DESCRIPTION OF THE DRAWINGS

These and other aspects will now be described in detail with reference to the following drawings. Generally, the figures are not to scale in absolute terms or comparatively, but are intended to be illustrative. Also, relative placement of features and elements may be modified for the purpose of illustrative clarity.

FIG. 1A is a diagram of the front portion of the eye;

FIG. 1B is a diagram of the layers of the eye;

FIG. 2 is a block diagram of an implementation of an ablation system;

FIG. 3 is an isometric view of an implementation of an ablation device;

FIG. 4A is an implementation of an ablation device having energy signal suppled from an external energy delivery generator by a cable;

FIG. 4B is an implementation of an ablation device having an attachable energy delivery generator;

FIG. 5A is a detailed view of an array incorporating rounded rods;

FIG. 5B is a detailed view of an array incorporated square-tipped rods;

FIG. 5C is a detailed view of an array incorporating a contour that substantially matches a surface contour of a portion of the eye;

FIG. 5D is a detailed view of an array incorporating extendable needles;

FIG. 6A is a perspective view of the device of FIG. 4A;

FIG. 6B is a cross-sectional view of the device of FIG. 6A taken along line B-B;

FIG. 7A is an exploded view from a back end perspective of the ablation device of FIG. 6A;

FIG. 7B is an exploded view from a front end perspective of the ablation device of FIG. 6A;

FIG. 8 is a close-up view of a distal end region of an implementation of a device incorporating an adjustable depth-setting collar and bipolar rod array;

FIG. 9 is a detailed view of an implementation of an ablation device.

It should be appreciated that the drawings are for example only and are not meant to be to scale. It is to be understood that devices described herein may include features not necessarily depicted in each figure.

### DETAILED DESCRIPTION

Disclosed are devices, systems, and methods for increasing aqueous humor outflow from the anterior chamber of an eye. More particularly and as will be described in detail below, provided is a tissue ablation end effector device using radiofrequency (RF) energy to enhance part of the natural drainage pathways of the eye. Radiofrequency ablation is a minimally-invasive technique. During treatment, microelectrodes are directly punctured into the target tissue and RF energy delivered through a rod. The local temperature is high and causes irreversible coagulating tissue autolysis. This is precisely controlled and generally non-invasive. The RF energy is used to enhance and/or create new drainage pathways of the eye for the purpose of lowering IOP.

The devices described herein apply the energy ab externo (i.e., from outside the eye) through the conjunctiva or under a conjunctival flap to create a non-penetrating trabeculectomy thereby reducing diffusion resistance and the outflow of aqueous humor from the anterior chamber. Thinning of the sclera can decrease IOP. At least some of the holes or cavities created by the device are non-penetrating in that they do not penetrate the full thickness of the sclera or enter the interior of the eye. In some implementations, the holes or cavities created only go as deep as the episcleral layer. In other implementations, the holes or cavities created can penetrate through the episcleral layer. The holes or cavities created can penetrate a portion of the sclera such as at least about 70% of the thickness, at least about 75%, at least about 80%, at least about 85%, at least about 90% of the thickness of the sclera and anywhere in between. At least one cavity formed by the rods 117 can be 100% a thickness of the sclera. The array 115 can include some rods 117 that have an exposed length sufficient to penetrate through the entire thickness of the sclera whereas other rods 117 in the array 115 may have a shorter exposed length that prevents them from penetrating through the entire thickness of the sclera and instead create partial thickness cavities. The partial thickness cavity can be deep enough within a tissue layer to leave a thin membrane layer of tissue between the outside of the eye and the inside of the eye so that aqueous humor can seep through the membrane layer into the cavity. The number and arrangement of rods 117 within a single array 115 that have an exposed length designed to penetrate the full thickness and the number and arrangement of rods 117 within the array 115 that have an exposed length designed to penetrate tissue less than full thickness can vary. In some implementations, all rods 117 within the array 115 have an exposed length designed to penetrate the full thickness and in other implementations, none of the rods 117 within the array 115 penetrate the full thickness. In still further implementations, some rods 117 within an array 115 may penetrate the full thickness of the sclera as well as one or more additional structures of the eye. For example, one or more rods 117 within the array 115 may have an exposed length sufficient to penetrate the full thickness of the sclera (with or without penetrating the conjunctival layer) and at least an outer wall of Schlemm's canal. One or more rods 117 within the array 115 may have an exposed length sufficient to penetrate the full thickness of the sclera (100% of the thickness), the outer wall of Schlemm's canal and across the canal to make contact with the trabecular meshwork. In this implementation, some of the rods 117 extend from outside the eye into the anterior chamber of the eye. Not all rods 117 need penetrate to the same depth or into the same sub-scleral structure.

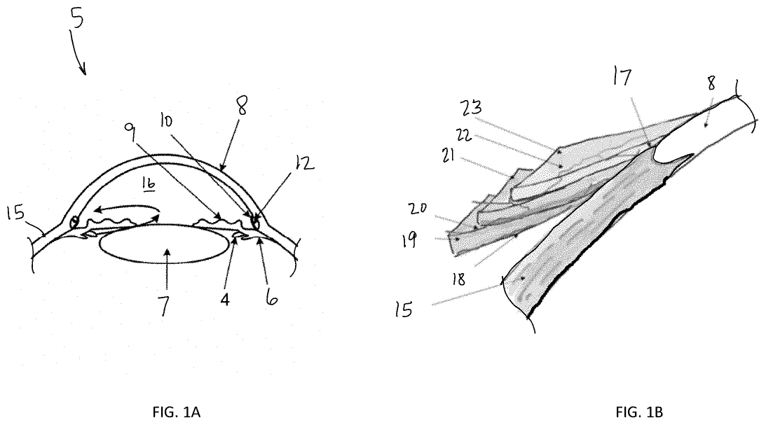

FIG. 1B illustrates the layers of the eye 5 near the limbus 17 including the sclera 15, the deep episcleral plexus 18, episclera 19, superficial episcleral plexus 20, Tenon's capsule 21, conjunctival plexus 22, and conjunctiva 23. The episclera 19 is the outermost layer of the sclera and includes loose, fibrous, elastic tissue and attaches to the Tenon's capsule 21. It lies directly between the clear outer conjunctiva 23 and the firm white part of the sclera beneath. The deep episcleral plexus 18 includes the layers of vessels including the deep episcleral vessels and the superficial episcleral plexus 20 includes the superficial episcleral vessels.

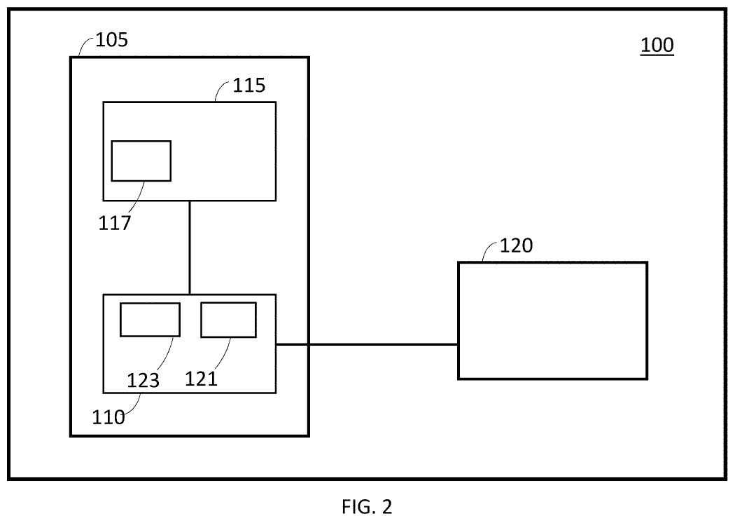

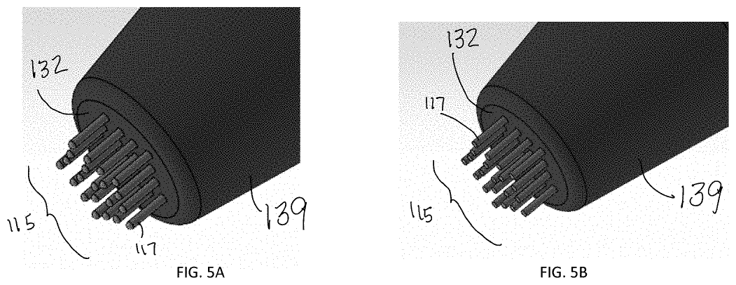

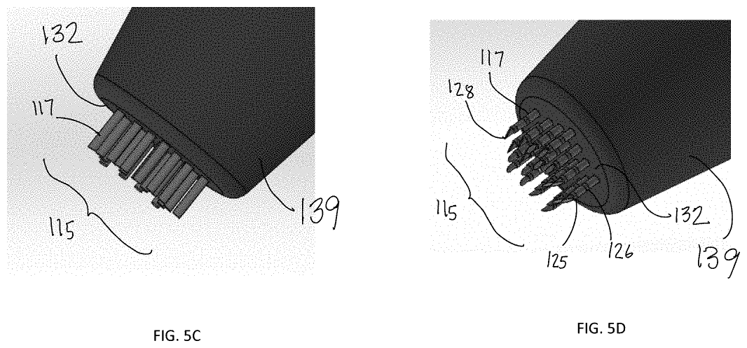

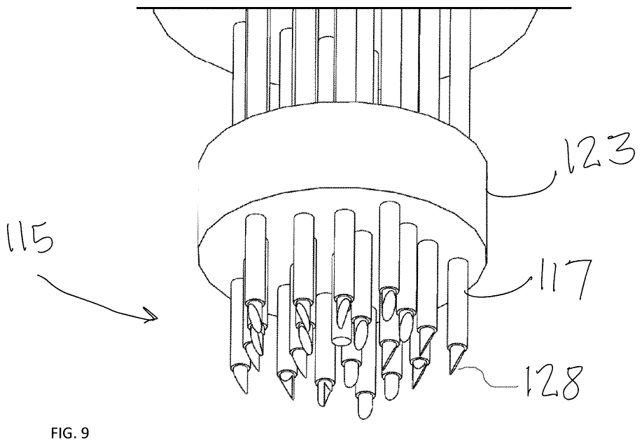

FIG. 2 is a block diagram of an implementation of an ablation system 100 for creating non-penetrating trabeculectomy to improve outflow of aqueous humor and reduction in TOP by reducing resistance to diffusion by the sclera. The system 100 includes a multi-pronged RF ablation device 105 having a handle 110 and an array 115 of electrically connected rods 117 projecting from a distal end region 119 of the handle 110. The device 105 can be electrically connected to an energy delivery generator 120. The energy delivery generator 120 can be an external generator coupled to the handle 110 as shown in FIG. 2. Alternatively, the RF signal can be provided by a reusable generator 120 positioned within at least a portion of the handle 110. The reusable generator 120 can be positioned within a reusable portion of the handle 110 that is designed to operatively couple with a disposable portion of the handle 110 as will be described in more detail below. The array 115 can use energy from the energy generator 120 to penetrate tissue or the array 115 can penetrate the tissue without energy active. The energy delivery generator 120 can be a monopolar or bipolar RF energy generator. With monopolar arrays, current passes from the electrode element to a dispersive electrode attached externally to the patient, e.g., using a contact pad placed on the patient's flank. With bipolar arrays, the RF current is delivered to two electrode elements in a bipolar fashion, which means that current will pass between “positive” and “negative” electrode elements. Bipolar arrangements require the RF energy to traverse through a relatively small amount of tissue between tightly spaced electrodes compared to monopolar arrangement that require the RF energy to traverse through the thickness of a patient's body. FIGS. 5D and 8 show implementations of the ablation device 105 having bipolar rods 117. Each bipolar rod 117 includes a bipolar anode 125 and a bipolar cathode 126. Thus, the array 115 contains two poles within a single rod 117. The generator 120 may be a conventional general purpose electrosurgical power supply (Bovie Box®, Symmetry Surgical, Nashville, Tenn.) operating at a frequency in the range of about 350 kHz and about 500 kHz, preferably about 490 kHz for both monopolar and bipolar. Output power can be set at about 50 W to about 300 W. The electric field strength can be between 250 and 4000 Volts. Monopolar voltage can be 1000 V or greater. Bipolar voltage can be under 1000 V. The energy delivery generator 120 can use RF as described in more detail below or another energy type including high voltage pulses, ultrasonic energy, or irreversible electroporation.

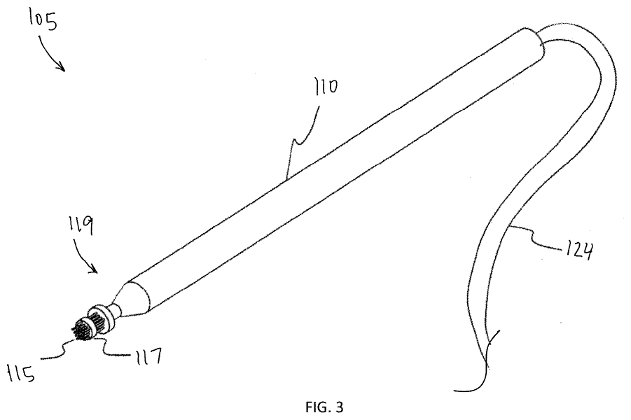

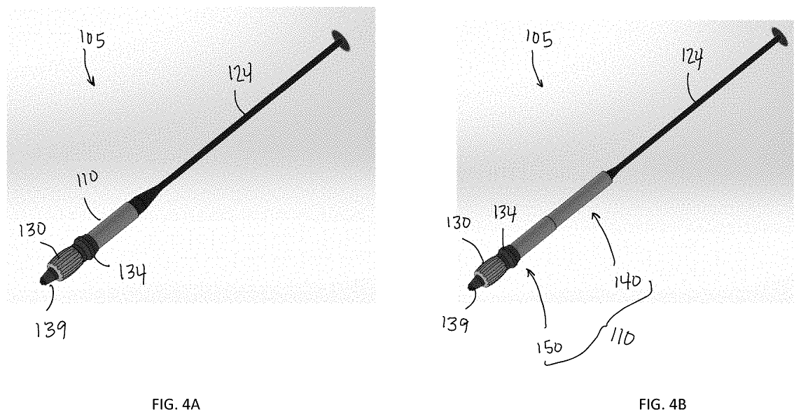

FIG. 3 is an isometric schematic view of an implementation of the ablation device 105. As mentioned above, the device 105 includes a handle 110 and an array 115. The energy delivery generator 120 in this implementation is an RF energy generator that is integrated within the handle 110. FIGS. 4A-4B are additional implementations of the ablation device 105. FIG. 4A shows the device includes a handle 110 that is supplied an RF signal from an external energy delivery generator (not shown) by a cable 124. FIG. 4B shows an ablation device 105 having an attachable RF generator within a reusable portion 140 of the handle 110 that is configured to operatively couple to a distal disposable portion 150 of the handle 110. The reusable portion 140 can couple to the distal disposable portion 150 using a variety of mechanisms such as threads, snap-lock, bayonet lock, and the like. In some implementations, the proximal end region of the disposable portion 150 can include a chamber having a proximal opening through which at least a portion of the durable portion 140 may be inserted for coupling to the disposable portion 150 such as via bayonet lock mechanism. The durable component 140 can include a bayonet connector with cable attached at a proximal end. The connection between the disposable portion 150 and durable portion 140 can be purely mechanical or both mechanical and electrical. The coupling between the disposable and durable portions can create a smooth continuous housing for the handle (see FIG. 4B).

Again with respect to FIGS. 4A-4B, the array 115 can include a plurality of electrically connected rods 117 projecting from the distal end region 119 of the handle 110. The rods 117 can be rounded in profile at their distal ends as shown in FIG. 5A or can be square-tipped in configuration as shown in FIG. 5B. The rods 117 can also have sharp tips that serve to increase the strength of the generated electric field at the tips or enable some non-ablative tissue penetration. The rods 117 can also incorporate needles 128 extending through them so that the rods 117 may have a first geometry at their distal tips and the needle 128 have a second, different geometry (see FIG. 5D). The number of rods 117 within the array can vary. The array 115 can include at least 10 rods 117 up to about 100 rods 117. The plurality of rods 117 of the array 115 can form a footprint having any of a variety of shapes for applying to the surface of the eye including square, rectangular, circular, oval, etc. The cross-section of each of the rods 117 in the array 115 may be circular, rectangular, triangular, hexagonal, or another shape. The area of the eye covered by the array 115 can vary as well. In some implementations, the area is sufficient span a band of the eye such as a single clock hour of the pars plana/pars plicata such as at about 1 o'clock. In some implementations, the size of the footprint (or the overall surface area of the eye that the array 115 covers) can vary from about 2 mm to about 15 mm in diameter.

The rods 117 can be fixed at various lengths relative to one another so that the array 115 forms a footprint having a distal concave contour that substantially matches or complements the surface or external convex contour of a portion of the eye against which it will be applied (see FIG. 5C). The contour or the diameter of the curve of the array 115 can be about 20 mm-25 mm, preferably between 23-24 mm in diameter and generally concave. The array 115 can have a single radius or multiple radii of curvature from about 10 mm to about 30 mm, about 12 mm to about 27 mm, preferably about 8 mm to about 12 mm. In an implementation, the array 115 can have a single radius of curvature that is between 10 mm and 15 mm. In another implementation, the array 115 can have multiple radii of curvature between 12 mm and 27 mm. The footprint of contact conforms generally to the shape or external convex contour of the eye near the limbus 17 when the longitudinal axis A of the handle 105 is aligned substantially perpendicular to the point of contact with the eye.

The limbus of the eye can serve as a reference point for placement of the array 115 against the eye. The rods 117 of the array 115 can be inserted through the conjunctival layer so that the conjunctival layer is left in place during use of the device. Alternatively, the conjunctival layer may be incised and a flap pulled back as in standard trabeculectomy or shifted with respect to the lower layers under the tool head. Pulling back or shifting the conjunctival layer exposes the sclera so that the rods 117 of the array 115 make contact with the scleral surface without penetrating through the conjunctival layer. In this methodology, following treatment of the scleral layer, the conjunctival layer may be returned to its original position over the treated scleral layer covering the cavities or holes created by the rods 117. Multiple holes or ablated cavities can be formed within the layers of the eye, such as the episclera 19, preferably over the location of the ciliary body 6 or within a selected distance from the limbus 17, such as about 2.0 mm to about 5 mm, or about 2.5 mm to about 4.5 mm, or about 3.0 mm to about 4.0 mm. In some implementations, the holes or ablated cavities can be placed over the pars plicata or pars plana. The systems described herein can incorporate a gauge tool or feature on one or more of the devices for achieving a desired distance from the limbus 17.

The array 115 can be applied to the eye one or more times for a single treatment. The rods 117 can be stationary or they can be movable. In some implementations, the rods 117 extend relative to the handle 110 in order to penetrate the tissue. In other implementations, the rods 117 retract relative to the handle 110. In still further implementations, the rods 117 both extend and retract relative to the handle 110. The rods 117 in a single array 115 can be the same length or different lengths in various places on the array 115, depending on the structures and thickness to be penetrated. The rods 117 can be stationary while another part of the handle 110 moves to change the relative extension of the rod 117 with respect to the handle 110 for penetration of the tissue. The rods 117 can be advanced distally in a smooth manner (as opposed to a fast trigger or injection punch). The advancement mechanism allows for the penetration of the tissue with minimal movement necessary by a surgeon to avoid unintentional movement and unintentional cutting. A surgeon can place the distal end of the device at a desired location on the eye and actuate the device to achieve a pre-set depth of penetration by the rods 117. In still further implementations, the rods 117 may be fixed relative to the handle 110 and incorporate needles extending through them that are movable. As an example, the surgeon could place the distal ends of the rods 117 against the eye and trigger the needles 128 extending through the rods 117 to advance distally to achieve a certain penetration depth. Sharpened tips of the needles 128 allow for penetrating the tissue before any RF energy is applied. Any of a variety of arrangements are considered herein.

In some implementations, the system can incorporate a depth sensing mechanism. The rods 117 may be actuated automatically until the system senses the correct depth via ultrasound, a physical depth sensor, or another means of proximity sensing of a marker or probe inserted into the eye. The marker can include a physical magnet that is surgically placed temporarily within a region of the eye such as within the anterior angle. Alternatively, the marker can be a fluid that is chemically, physically, magnetically, or electronically sensed by the system. The fluid can be injected into a region of the eye such as Schlemm's canal, the collector channels, or another space of the eye adjacent the target treatment site. In some implementations, one or more of the rods of the array of rods can be a depth sensor. One or more of the rods in the array of rods also can be hollow and usable as a flashback indicator for detecting a liquid during use of the device.

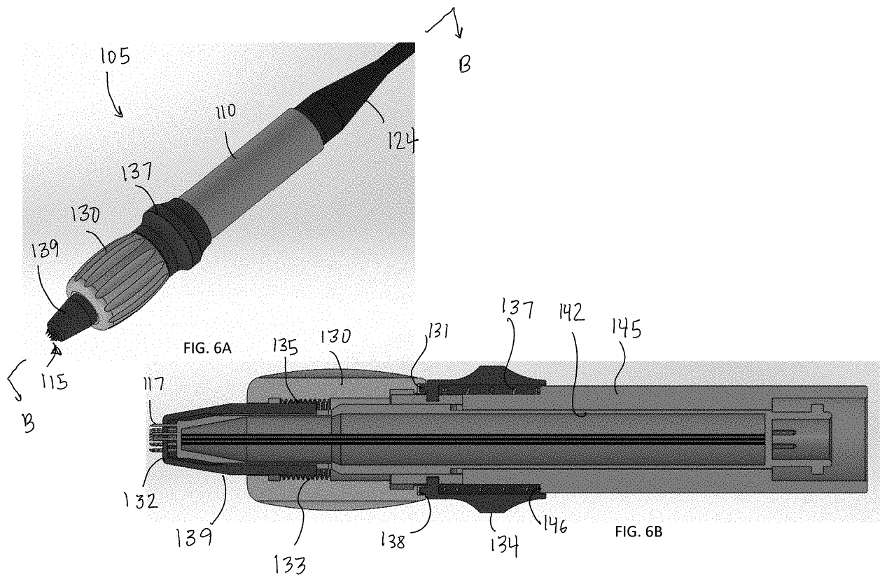

As mentioned above, the rods 117 are designed to create holes within the sclera that are less than full thickness or non-penetrating holes or ablated cavities. The maximum extended length of the rods 117 can range from about 0.3 mm to about 0.8 mm, preferably about 0.5 mm. The device 105 can additionally incorporate a collar or other adjustable depth-setting mechanism 123 configured to control depth of insertion of the array 115 (see, e.g., FIGS. 6A-6B and also FIG. 8). The mechanism 123 can be adjusted such as with a leadscrew, friction slider, electromagnetic actuator, and the like to control the depth of insertion of the rod array 115. The mechanism 123 can be adjusted depending on whether the array 115 is to be inserted under or pierced through the conjunctival flap. The depth of penetration is variable and can be set as a percentage of overall scleral thickness within the treatment zone. The mechanism 123 can limit the distance the rods 117 project outside the handle 110. In some implementations, the rods 117 are already extended and in other implementations the rods 117 are extended using an actuator.

FIG. 6A is a perspective view of the ablation device of FIG. 4A and FIG. 6B is a cross-sectional view of the device taken along line B-B illustrating an implementation of the adjustable depth-setting mechanism 123. The mechanism 123 can vary in structure. In this implementation, the mechanism 123 includes an actuator such as a depth adjustment knob 130 encircling a distal end region of the handle 110 that is coupled to a depth setting plate 132. The actuator is configured to incrementally change the position of the plate 132. The mechanism 123 can further include a locking mechanism 134 such as a locking ring configured to fix a position of the plate 132. The depth setting plate 132 has a plurality of openings 136 extending through its thickness. Each opening 136 is sized and shaped to receive each rod 117 of the array so that the array of rods projects a distance distal to the plate 132. The rods 117 extending through the handle 110 of the device 105 are configured to project through the plate 132 a distance depending on the position of the plate 132 relative to the distal end region of the handle 110. A position of the plate relative to the distal end region of the handle sets a length of each rod of the array of rods that is exposed distal to the plate 132. Actuating the actuator to advance the depth setting plate 132 distally reduces an exposed length of each rod thereby limiting a depth of penetration achievable by the rods. Activating the actuator to retract the depth setting plate proximally increases the exposed length of the rods and thereby maximizes the depth of penetration achievable by the rods. The plate 132 can be include a proximal threaded region 133 that engages with corresponding threads 135 on the inner surface of the depth adjustment knob 130 encircling the handle 110. The engagement between the threaded region 133 and the threads 135 of the knob 130 allows for the plate 132 to be advanced and retracted relative to the rods 117. The plate 132 is advanced distally to decrease the depth of penetration of the plurality of rods 117 because this effectively reduces the length of the rods 117 outside the handle 110. The plate 132 is retracted proximally to increase the depth of penetration of the plurality of rods 117 because the length of the rods 117 extending outside the handle 110 is increased. The rods 117 can be automatically advanced by the system such as when a user triggers the rods 117 to advance. The rods 117 can also be manually advanced by the user. Whether the rods are advanced by the system or by the user, the desired depth of penetration achieved by the rods 117 or needles upon actuation can be set such as by mechanical actions that are manual or automated by the system.

Still with respect to FIGS. 6A-6B, the locking ring 134 can toggle back and forth to allow and prevent, respectively, the movement of the plate 132. The locking ring 134 can be biased by a spring 137 toward a distal end of the handle 110. When urged distally, the locking ring 134 engages with a proximal end of the knob 130 preventing it from retracting the plate 132 thereby fixing the plate position and depth of penetration that is possible. The locking ring 134 can be retracted compressing the spring 137 and disengaging the ring 134 from the knob 130 so as to allow the adjustment of the plate 132 position relative to the rods 117. Once the desired extension of the rods 117 is achieved, the locking ring 134 can be released and the spring 137 once again urges it into engagement with the knob 130 thereby locking the position of the plate 132 relative to the rods 117.

The array 115 may use energy to penetrate tissue or may be inserted without energy active. The ablation device 105 can also include extendable needles 128 (see, e.g., FIG. 5D and FIG. 9). The array 115 having the extendable needles 128 may be useful for tissue penetration or for the deposition of substances into the ablated cavity or unablated tissue. Each of the electrically connected rods 117 of the array 115 can include a needle 128 that is movable within the rod 117. The needles can be advanced using mechanical means that can be electrically driven, pneumatically, by an applied external force by the user or by stored mechanical energy. This actuation may be fast or slow. The needles may be advanced through the center of each rod 117, or the needles may be advanced over the outside of each rod 117 so that the rods 117 extend through the center of the needles. The needles may stay in place during the RF activation, or they may be moved so that the rods 117 are exposed during RF activation. Still further, the rods 117 may have a central bore (e.g., that can be used to hold a needle 128 or left open) that can allow for fluid or another substance to be injected before, during, and/or after the ablation process. Substances such as viscoelastic or one or more therapeutic agents (e.g., anti-metabolite antineoplastic agents such as 5-fluorouracil (5-FU) or anti-inflammatories, etc.), or physical structures that may or may not elute substances over time may also be injected through the central hole in the rods 117 or through needles 128 positioned within the rods 117. The surgeon can position the device against the eye, such as by placing the distal tips of the rods 117, and trigger the needles 128 to deploy into the tissue by extending distally to project out the distal tip of the rods 117.

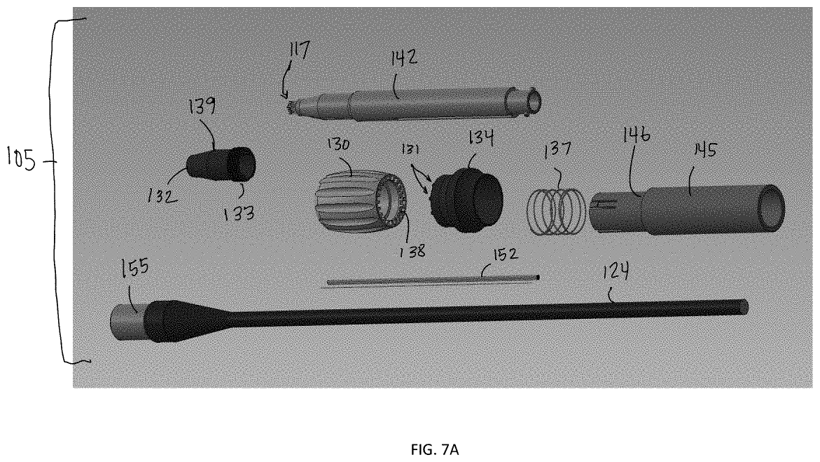

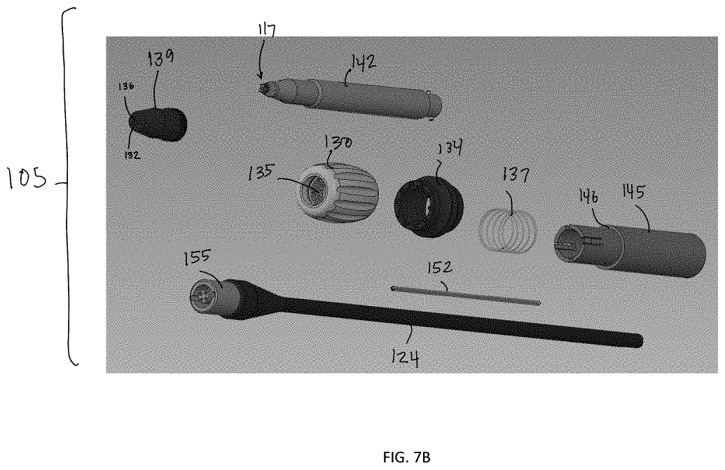

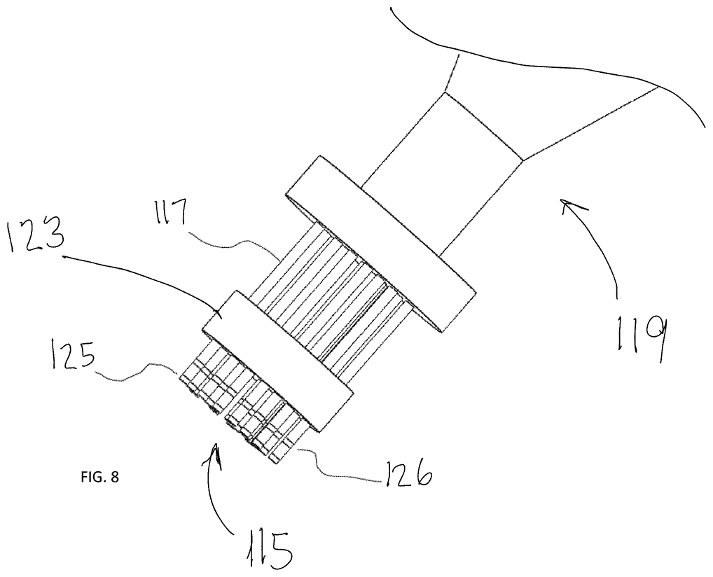

Again with respect to FIGS. 6A-6B as well as FIGS. 7A-7B, the device 105 can include a depth-setting mechanism 123 as discussed above that includes a depth adjustment knob 130, an end cap 139 having the depth setting plate 132 having proximal threaded region 133, a locking ring 134, and spring 137. The locking ring 134 and knob 130 have inner diameters that are sized to receive an outer diameter of an inner body 142. The locking ring 134 encircles a first region of the inner body 142 just proximal to a second region of the inner body 142 encircled by the knob 130. A distal end region of the locking ring 134 engages with a proximal end region of the knob 130. The distal end region of the locking ring 134 can include one or more teeth 131 sized and shaped to be received within indentations 138 on an inner surface of the proximal end region of the knob 130. As the knob 130 turns around the long axis of the inner body 142 so too does the locking ring 134. The end cap encircles the distal end region of the inner body 142 having the plurality of rods 117 projecting distally from the distal-most end 143 of the inner body 142. The handle 110 also includes a proximal shell 145. The spring 137 can be received over the distal end region of the shell 145. The distal end region of the shell 145 can provide a backstop 146 for the spring 137 to abut up against for compressing the spring 137 within the handle 110. A cable bundle 152 extends through the interior of the inner body 142 and proximal shell 145 to connect between the rods 117 and the cable receptacle 155. The cable receptacle 155 is detachable from the proximal end of the shell 145 as discussed elsewhere herein.

The cable 124 can be a standard RF cable configured to provide RF power to the device 105 from an external RF generator 120. Alternatively, the RF generator 120 can be within the handle 110 of the device 105 and the cable 124 be configured to provide power to the generator 120. The RF current can be delivered to the array 115.

Power can be supplied by a power system of the system 100 when the device 105 is operatively coupled to the system 100. The device 105 can include a cable extending from the durable portion. The cable may also be configured to connect the device 105 to a wall socket. The device 105 can also be powered by one or more batteries. The battery can be incorporated within a region of the durable portion, either internally or coupled to a region of the housing such as within a modular, removable battery pack. The battery can have different chemical compositions or characteristics. For instance, batteries can include lead-acid, nickel cadmium, nickel metal hydride, silver-oxide, mercury oxide, lithium ion, lithium ion polymer, or other lithium chemistries. The device can also include rechargeable batteries using either a DC power-port, induction, solar cells, or the like for recharging. Power systems known in the art for powering medical devices for use in the operating room are also to be considered herein such as spring power or any other suitable internal or external power source.

The array can be connected to other types of energy delivery generators than radiofrequency, including an energy delivery generator that uses high voltage pulses, an ultrasonic energy generator, or an energy delivery generator using irreversible electroporation.

One or more components of the system can be controlled by a computer unit powered by a power system. The computing unit can include a control processor, memory, storage devices, interconnected by a system bus. The memory is configured for receiving and storing user input data. The memory can be any type of memory capable of storing data and communication that data to one or more other components of the system, such as the control processor. The memory may be one or more of a Flash memory, SRAM, ROM, DRAM, RAM, EPROM, dynamic storage, and the like. The memory can be configured to store one or more user-defined profiles relating to the intended use of the instrument. The memory can be configured to store user information, history of use, and the like.

The communication module of the computing unit can be in operative communicate with one or more components of the system, such as the control processor, as well as with one or more peripheral devices. The connection can include a wired communication port such as a RS22 connection, USB, Fire wire connections, proprietary connections, or any other suitable type of hard-wired connection configured to receive and/or send information to an external computing device or ablation device. The communication module can also include a wireless communication port such that information can be fed between the computing unit and the external computing device and/or device via a wireless link, for example, to display information in real-time on the external computing device about operation of the system, and/or control programming of the ablation device. It should be appreciated that the external computing device, such as a console or tablet, can communicate directly to the ablation device. Any of a variety of adjustments to and programming of the system can be performed using the external computing device. The wireless connection can use any suitable wireless system, such as Bluetooth, Wi-Fi, radio frequency, ZigBee communication protocols, infrared, or cellular phone systems, and can also employ coding or authentication to verify the origin of the information received. The wireless connection can also be any of a variety of proprietary wireless connection protocols.

The system can include a control unit, power source, microprocessor computer, and the like. Aspects of the subject matter described herein may be realized in digital electronic circuitry, integrated circuitry, specially designed ASICs (application specific integrated circuits), computer hardware, firmware, software, and/or combinations thereof. These various implementations may include an implementation in one or more computer programs that are executable and/or interpretable on a programmable system including at least one programmable processor, which may be special or general purpose, coupled to receive signals, data and instructions from, and to transmit signals, data, and instructions to, a storage system, at least one input device, and at least one output device.

These computer programs (also known as programs, software, software applications, or code) include machine instructions for a programmable processor, and may be implemented in a high-level procedural and/or object-oriented programming language, and/or in assembly/machine language. As used herein, the term “machine-readable medium” refers to any computer program product, apparatus, and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

The device 105 can include one or more actuators 121 on the handle 110 for automatic actuation of the needles and control of the RF box. The actuator 121 can be on the reusable portion 140 or the disposable portion 150 of the handle 110. The configuration of the actuator(s) 121 can vary. For example, the actuator(s) 121 can include a lead screw system with a motor or a mechanical system that uses stored energy to turn a screw and advance the needles. The actuator(s) 121 can also incorporate a pneumatic system using air or another gas to move the needles distally. The actuator(s) 121 can include one or more triggers, buttons, sliders, dials, keypads, switches, touchscreens, foot pedals, or other input that can be retracted, pressed, squeezed, slid, tapped, or otherwise actuated to activate, modify, or otherwise cause a response of the device 105. The actuator can also be remote from the handle in a wired or wireless manner. The device may include one or more outputs such as lights, speakers, vibration motors, displays or other sort of output configured to communicate information to the user by visual, audio, and/or tactile outputs.

The rods 117 are preferably formed of conductive materials such as biocompatible metals or, in the case of a bipolar array, a conductive material separated by a non-conductive material such as plastic or ceramic. The handle can be a medical grade plastic or metal.

In some implementations, a guide tool designed to stabilize the distal end region and, in particular the array, during application of energy to the eye. The guide tool can be a Thornton Fixation Ring having a handle, a swivel head defining an internal diameter, and a plurality of blunt teeth on an underside of the head. The blunt teeth can be used to stabilize and/or rotate the globe during a procedure. The distal end region of the ablation device 105 can be positioned on a region of the head, or even interlocked with the head for stability during use.

In various implementations, description is made with reference to the figures. However, certain implementations may be practiced without one or more of these specific details, or in combination with other known methods and configurations. In the description, numerous specific details are set forth, such as specific configurations, dimensions, and processes, in order to provide a thorough understanding of the implementations. In other instances, well-known processes and manufacturing techniques have not been described in particular detail in order to not unnecessarily obscure the description. Reference throughout this specification to “one embodiment,” “an embodiment,” “one implementation, “an implementation,” or the like, means that a particular feature, structure, configuration, or characteristic described is included in at least one embodiment or implementation. Thus, the appearance of the phrase “one embodiment,” “an embodiment,” “one implementation, “an implementation,” or the like, in various places throughout this specification are not necessarily referring to the same embodiment or implementation. Furthermore, the particular features, structures, configurations, or characteristics may be combined in any suitable manner in one or more implementations.

The use of relative terms throughout the description may denote a relative position or direction. For example, “distal” may indicate a first direction away from a reference point. Similarly, “proximal” may indicate a location in a second direction opposite to the first direction. The reference point used herein may be the operator such that the terms “proximal” and “distal” are in reference to an operator using the device. A region of the device that is closer to an operator may be described herein as “proximal” and a region of the device that is further away from an operator may be described herein as “distal”. Similarly, the terms “proximal” and “distal” may also be used herein to refer to anatomical locations of a patient from the perspective of an operator or from the perspective of an entry point or along a path of insertion from the entry point of the system. As such, a location that is proximal may mean a location in the patient that is closer to an entry point of the device along a path of insertion towards a target and a location that is distal may mean a location in a patient that is further away from an entry point of the device along a path of insertion towards the target location. However, such terms are provided to establish relative frames of reference, and are not intended to limit the use or orientation of the devices to a specific configuration described in the various implementations.

As used herein, the term “about” means a range of values including the specified value, which a person of ordinary skill in the art would consider reasonably similar to the specified value. In aspects, about means within a standard deviation using measurements generally acceptable in the art. In aspects, about means a range extending to +/−10% of the specified value. In aspects, about includes the specified value.

While this specification contains many specifics, these should not be construed as limitations on the scope of what is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results. Only a few examples and implementations are disclosed. Variations, modifications and enhancements to the described examples and implementations and other implementations may be made based on what is disclosed.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.”

Use of the term “based on,” above and in the claims is intended to mean, “based at least in part on,” such that an unrecited feature or element is also permissible.

The systems disclosed herein may be packaged together in a single package. The finished package would be sterilized using sterilization methods such as Ethylene oxide or radiation and labeled and boxed. Instructions for use may also be provided in-box or through an internet link printed on the label.

All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of any claims. No language in the specification should be construed as indicating any non-claimed element essential to the practice of the invention.

Groupings of alternative elements, embodiments, or implementations disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

### P EMBODIMENTS

P Embodiment 1. A multi-pronged RF ablation device comprising: an array of electrically connected rods; a depth setting plate; a handle; and a cord.

P Embodiment 2. The multi-pronged ablation device of P Embodiment 1 where the rods in the array of electrically connected rods are square tipped in profile.

P Embodiment 3. The multi-pronged ablation device of P Embodiment 1 or 2 where the rods in the array of electrically connected rods are rounded in profile.

P Embodiment 4. The multi-pronged ablation device of any one of P Embodiments 1-3 where the rods in the array of electrically connected rods are fixed at various lengths to match the contour of the surface of the sclera.

P Embodiment 5. The multi-pronged ablation device of any one of P Embodiments 1˜4 where the array of electrically connected rods is connected to a monopolar RF generator.

P Embodiment 6. The multi-pronged ablation device of any one of P Embodiments 1-5 where the array of electrically connected rods is connected to a bipolar RF generator.

P Embodiment 7. The multi-pronged ablation device of any one of P Embodiments 1-6 where the array of electrically connected rods contains two poles within a single needle.

P Embodiment 8. The multi-pronged ablation device of any one of P Embodiments 1-7 where the depth setting plate is controlled by a leadscrew.

P Embodiment 9. The multi-pronged ablation device of any one of P Embodiments 1-8 where the RF generator is integrated into the handle.

P Embodiment 10. The multi-pronged ablation device of any one of P Embodiments 1-9 where the rods in the array of electrically connected rods contain retractable needles within each rod.

P Embodiment 11. The multi-pronged ablation device of any one of P Embodiments 1-10 where the array of electrically connected rods is connected to an energy delivery generator utilizing high voltage pulses.

P Embodiment 12. The multi-pronged ablation device of any one of P Embodiments 1-11 where the array of electrically connected rods is connected to an energy delivery generator utilizing high voltage pulses.

P Embodiment 13. The multi-pronged ablation device of any one of P Embodiments 1-12 where the array of electrically connected rods is connected to an ultrasonic energy generator.

P Embodiment 14. The multi-pronged ablation device of any one of P Embodiments 1-13 where the array of electrically connected rods is connected to an energy delivery generator utilizing irreversible electroporation.

1. An ablation device for the treatment of an eye to lower intraocular pressure, the device comprising:
a handle; and
an array of rods projecting from a distal end region of the handle and electrically-connected to an energy delivery generator, wherein each rod of the array of rods is configured to create a cavity in a surface of the eye via ab externo tissue ablation to enhance drainage of aqueous humor from the eye.

2. The device of claim 1, wherein the surface of the eye includes the sclera.

3. The device of claim 2, wherein each cavity has a depth that is less than a full thickness of the sclera.

4. The device of claim 3, wherein each cavity is at least about 70% a thickness of the sclera up to about 90% the thickness of the sclera.

5. The device of claim 2, wherein at least one cavity is 100% a thickness of the sclera.

6. The device of claim 2, wherein resistance to diffusion by the sclera is lowered.

7. The device of claim 1, wherein each rod of the array of rods is square-tipped or rounded in profile.

8. The device of claim 1, wherein a cross-section of each rod of the array of rods is rectangular, circular, triangular, or hexagonal.

9. The device of claim 1, wherein the array of rods is designed to penetrate tissue without applying energy from the energy delivery generator.

10. The device of claim 1, wherein each rod of the array of rods is fixed relative to the distal end region of the handle so that the array of rods forms a footprint having a distal concave contour that complements an external convex contour of a portion of the eye being treated.

11. The device of claim 10, wherein the footprint of the array conforms generally to the external convex contour when a longitudinal axis of the handle is aligned perpendicular to a point of contact with the eye.

12. The device of claim 1, further comprising a gauge tool or gauge feature near a distal end of the device to align the array a selected distance from the limbus of the eye.

13. The device of claim 1, further comprising a depth-setting mechanism configured to control depth of insertion of the array.

14. The device of claim 13, wherein the depth-setting mechanism is adjustable.

15. The device of claim 14, wherein the depth-setting mechanism includes an actuator that is coupled to a depth setting plate.

16. The device of claim 15, wherein the depth setting plate comprises a plurality of openings extending through its thickness, each opening of the plurality of openings is sized and shaped to receive each rod of the array of rods so that the array of rods projects a distance distal to the plate.

17. The device of claim 16, wherein a position of the plate relative to the distal end region of the handle sets a length of each rod of the array of rods that is exposed distal to the plate.

18. The device of claim 17, wherein the actuator is configured to incrementally change the position of the plate.

19. The device of claim 18, wherein actuating the actuator to advance the depth setting plate distally reduces an exposed length of each rod and wherein actuating the actuator to retract the depth setting plate proximally increases an exposed length of each rod.

20. The device of claim 15, wherein the depth setting mechanism further comprises a locking mechanism configured to fix a position of the plate.

21. The device of claim 1, wherein each rod has an exposed length available to extend within the surface of the eye.

22. The device of claim 21, wherein the exposed length is greater than a thickness of the sclera of the eye.

23. The device of claim 21, wherein the exposed length is adjustable.

24. The device of claim 1, wherein one or more rods of the array of rods is hollow and usable as a flashback indicator for detecting liquid.

25. The device of claim 1, wherein one or more rods of the array of rods is a depth sensor.

26. The device of claim 1, wherein the energy delivery generator is an external generator coupled to the handle by a cable.

27. The device of claim 1, wherein the energy delivery generator is integrated into the handle.

28. The device of claim 27, wherein the energy delivery generator is a radio frequency (RF) generator.

29. The device of claim 27, wherein the energy delivery generator is integrated within a proximal, reusable portion of the handle, the reusable portion of the handle designed to operatively couple with a distal, disposable portion of the handle.

30. The device of claim 27, wherein the proximal, reusable portion and distal, disposable portion couple together by threads, snap-lock, or bayonet lock.

31. The device of claim 1, wherein the energy delivery generator is a monopolar RF generator.

32. The device of claim 1, wherein the energy delivery generator is a bipolar RF generator.

33. The device of claim 32, wherein each rod of the array of rods contains two poles.

34. The device of claim 1, wherein the energy delivery generator operates at a frequency in the range of about 350 kHz to 500 kHz.

35. The device of claim 34, wherein the energy delivery generator operates at 490 kHz.

36. The device of claim 31, wherein the monopolar voltage is 1000V or more.

37. The device of claim 32, wherein the bipolar voltage is under 1000V.

38. The device of claim 1, wherein the energy delivery generator uses high voltage pulses.

39. The device of claim 1, wherein the energy delivery generator is an ultrasonic energy generator.

40. The device of claim 1, wherein the energy delivery generator uses irreversible electroporation.

41. The device of claim 1, wherein each rod of the array is stationary.

42. The device of claim 1, wherein each rod of the array is movable relative to the handle so as to extend distally to penetrate tissue upon actuation.

43. The device of claim 1, wherein each rod of the array of rods is hollow for deposition of a substance into the cavity.

44. The device of claim 1, wherein each rod of the array of rods is automatically advanced to a fixed target depth.

45. The device of claim 1, wherein each rod of the array of rods is automatically advanced to a depth determined by a sensor of the device.

46. The device of claim 45, wherein the sensor is a physical depth sensor or an ultrasound-enabled depth sensor.

47. The device of claim 1, further comprising a plurality of extendable needles, each rod of the array comprising an extendable needle.

48. An ablation system for the treatment of an eye to lower intraocular pressure, the system comprising:
an energy delivery generator;
a handle; and
an array of rods projecting from a distal end region of the handle and electrically-connected to the energy delivery generator, wherein each rod of the array of rods is configured to create a cavity in a surface of the eye via ab externo tissue ablation to enhance drainage of aqueous humor from the eye.

49. The system of claim 48, wherein the energy delivery generator is an external generator coupled to the handle by a cable.

50. The system of claim 48, wherein the energy delivery generator is integrated into the handle.

51. The system of claim 48, wherein the energy delivery generator is a radio frequency (RF) generator.

52. The system of claim 48, wherein the energy delivery generator is integrated within a proximal, reusable portion of the handle, the reusable portion of the handle designed to operatively couple with a distal, disposable portion of the handle.

53. The system of claim 52, wherein the distal, disposable portion comprises the array of rods.
